(19) United States
(12) Patent Application Publication
(10) Pub. No.: US 2024/0418694 A1
(43) Pub. Date: Dec. 19, 2024

(54) ELECTRICAL PLANT MONITORING DEVICE AND OPERATION METHOD THEREOF

(71) Applicants: ELECTRONICS AND TELECOMMUNICATIONS RESEARCH INSTITUTE, Daejeon (KR); Korea Advanced Institute Of Science And Technology, Daejeon (KR)

(72) Inventors: JAE JOON KIM, Daejeon (KR); Seongjun PARK, Daejeon (KR); Chan Woo PARK, Daejeon (KR); Sungha JEON, Daejeon (KR); Min-Soo CHOI, Daejeon (KR); Ji-won HONG, Daejeon (KR); Kiwook HWANG, Daejeon (KR)

(73) Assignees: ELECTRONICS AND TELECOMMUNICATIONS RESEARCH INSTITUTE, Daejeon (KR); Korea Advanced Institute Of Science And Technology, Daejeon (KR)

(21) Appl. No.: 18/741,515

(22) Filed: Jun. 12, 2024

(30) Foreign Application Priority Data
Jun. 14, 2023 (KR) 10-2023-0076229
May 13, 2024 (KR) 10-2024-0062414

(51) Int. Cl. G01N 33/00; A01G 7/04; G01N 27/02

(52) U.S. Cl. CPC G01N 33/0098; A01G 7/04; G01N 27/028

(57) ABSTRACT

Disclosed is an electrical plant monitoring device which includes a plurality of flexible electrodes that are attached to a plate-shaped tissue and a tubular tissue of a plant, an impedance measurement unit that obtains electrical signals by using the plurality of flexible electrodes and measures impedance values associated with the plate-shaped tissue and the tubular tissue based on the electrical signals, and a spectrum monitor that generates an impedance spectrum based on the impedance values and monitors the impedance spectrum to sense an ion stress of the plant.

### CROSS-REFERENCE TO RELATED APPLICATIONS

This application claims priority under 35 U.S.C. § 119 to Korean Patent Application Nos. 10-2023-0076229 filed on Jun. 14, 2023, and 10-2024-0062414 filed on May 13, 2024, in the Korean Intellectual Property Office, the disclosures of which are incorporated by reference herein in their entireties.

### BACKGROUND

Embodiments of the present disclosure described herein relate to an electrical plant monitoring device and an operation method thereof, and more particularly, relate to an electrical plant monitoring device sensing an ion stress of a plant and an operation method thereof.

A conventional plant health monitoring technology measures the ion concentration of the plant in an external environment such as a soil or a nutrient solution, monitors a single stress associated with a water content, or uses an invasive method. However, the methods are incapable of directly measuring a change in the plant according to ions, incapable of monitoring various stresses, and capable of damaging the plant.

Accordingly, there is required a method for measuring the ion stress of the plant itself while minimizing the damage to the plant.

### SUMMARY

Embodiments of the present disclosure provide an electrical plant monitoring device sensing an ion stress of a plant and an operation method thereof.

According to an embodiment, an electrical plant monitoring device includes a plurality of flexible electrodes that are attached to a plate-shaped tissue and a tubular tissue of a plant, an impedance measurement unit that obtains electrical signals by using the plurality of flexible electrodes and measures impedance values associated with the plate-shaped tissue and the tubular tissue based on the electrical signals, and a spectrum monitor that generates an impedance spectrum based on the impedance values and monitors the impedance spectrum to sense an ion stress of the plant.

As an example, the plurality of flexible electrodes include a first flexible electrode pair attached to the plate-shaped tissue, and a second flexible electrode pair attached to the tubular tissue, and the impedance measurement unit obtains a first electrical signal among the electrical signals by using the first flexible electrode pair and obtains a second electrical signal among the electrical signals by using the second flexible electrode pair.

As an example, the impedance measurement unit measures a first impedance value between flexible electrodes of the first flexible electrode pair from among the impedance values, based on the first electrical signal and measures a second impedance value between flexible electrodes of the second flexible electrode pair from among the impedance values, based on the second electrical signal.

As an example, the impedance measurement unit includes a voltage supply unit that supplies a voltage to each of the first flexible electrode pair and the second flexible electrode pair, a signal reception unit that receives the first electrical signal, which is based on the voltage, from the first flexible electrode pair and receives the second electrical signal, which is based on the voltage, from the second flexible electrode pair, and a measurement unit that measures the first impedance value based on the first electrical signal and measures the second impedance value based on the second electrical signal.

As an example, the signal reception unit includes a filter that removes a noise included in the first electrical signal and the second electrical signal and outputs a first filter signal and a second filter signal which are noise-free and an amplifier that amplifies the first filter signal and the second filter signal respectively to output a first amplification signal and a second amplification signal, and the measurement unit measures the first impedance value from the first amplification signal and measures the second impedance value from the second amplification signal.

As an example, the voltage supply unit adjusts the voltage such that a frequency of the voltage changes. As a result of monitoring the impedance spectrum, the spectrum monitor senses the ion stress by analyzing a change in the first impedance value and a change in the second impedance value corresponding to the change in the frequency.

As an example, the plurality of flexible electrodes further include a third flexible electrode pair attached to the plate-shaped tissue. The impedance measurement unit obtains a third electrical signal among the electrical signals by using the third flexible electrode pair. The flexible electrodes included in the first flexible electrode pair and the third flexible electrode pair are arranged to be spaced apart from each other by a given distance.

As an example, a transfer path of the ion stress is inferred based on the first flexible electrode pair and the third flexible electrode pair.

As an example, a transfer path of the ion stress is inferred by adjusting a distance of flexible electrodes of the second flexible electrode pair.

According to an embodiment, an operation method of an electrical plant monitoring device includes supplying, at an impedance measurement unit, a voltage to a plurality of flexible electrodes attached to a plate-shaped tissue and a tubular tissue of a plant, measuring, at the impedance measurement unit, impedance values associated with the plate-shaped tissue and the tubular tissue based on the voltage, generating, at a spectrum monitor, an impedance spectrum based on the impedance values, and monitoring, at the spectrum monitor, the impedance spectrum to sense an ion stress of the plant.

As an example, the plurality of flexible electrodes include a first flexible electrode pair attached to the plate-shaped tissue and a second flexible electrode pair attached to the tubular tissue.

As an example, the measuring of the impedance values associated with the plate-shaped tissue and the tubular tissue based on the voltage at the impedance measurement unit includes measuring, at the impedance measurement unit, a first impedance value between flexible electrodes of the first flexible electrode pair by using the first flexible electrode pair, and measuring, at the impedance measurement unit, a second impedance value between flexible electrodes of the second flexible electrode pair by using the second flexible electrode pair.

As an example, the measuring of the first impedance value between the flexible electrodes of the first flexible electrode pair by using the first flexible electrode pair at the impedance measurement unit includes obtaining, at the impedance measurement unit, a first electrical signal by using the first flexible electrode pair, and measuring, at the impedance measurement unit, the first impedance value based on the first electrical signal. The measuring of the second impedance value between the flexible electrodes of the second flexible electrode pair by using the second flexible electrode pair at the impedance measurement unit includes obtaining, at the impedance measurement unit, a second electrical signal by using the second flexible electrode pair, and measuring, at the impedance measurement unit, the second impedance value based on the second electrical signal.

As an example, the obtaining of the first electrical signal by using the first flexible electrode pair at the impedance measurement unit includes removing, at the impedance measurement unit, a noise included in the obtained first electrical signal, and amplifying the noise-removed first electrical signal. The obtaining of the second electrical signal by using the second flexible electrode pair at the impedance measurement unit includes removing, at the impedance measurement unit, a noise included in the obtained second electrical signal, and amplifying the noise-removed second electrical signal.

As an example, the supplying of the voltage to the plurality of flexible electrodes attached to the plate-shaped tissue and the tubular tissue of the plant at an impedance measurement unit includes supplying, at the impedance measurement unit, a first voltage with a first frequency, and after supplying the first voltage, supplying, at the impedance measurement unit, a second voltage with a second frequency.

As an example, the measuring of the impedance values associated with the plate-shaped tissue and the tubular tissue based on the voltage at the impedance measurement unit includes measuring, at the impedance measurement unit, a first impedance value and a second impedance value, which are based on the first voltage, and measuring, at the impedance measurement unit, a third impedance value and a fourth impedance value, which are based on the second voltage. The first impedance value and the third impedance value are associated with the plate-shaped tissue, and the second impedance value and the fourth impedance value are associated with the tubular tissue.

As an example, the monitoring of the impedance spectrum to sense the ion stress of the plant at the spectrum monitor includes analyzing a difference of the first impedance value and the third impedance value according to a difference of the first frequency and the second frequency, and analyzing a difference of the second impedance value and the fourth impedance value according to the difference of the first frequency and the second frequency.

As an example, the monitoring of the impedance spectrum to sense the ion stress of the plant at the spectrum monitor includes inferring, at the spectrum monitor, a transfer path of the ion stress.

As an example, the inferring of the transfer path of the ion stress at the spectrum monitor includes adjusting a distance between the plurality of flexible electrodes.

### BRIEF DESCRIPTION OF THE FIGURES

The above and other objects and features of the present disclosure will become apparent by describing in detail embodiments thereof with reference to the accompanying drawings.

FIG. 1 illustrates an electrical plant monitoring device according to an embodiment of the present disclosure.

FIG. 2 illustrates an impedance measurement unit of FIG. 1 in detail.

FIGS. 3A to 3D illustrate an example of an operation of an electrical plant monitoring device of FIG. 1.

FIG. 4 illustrates a signal reception unit of FIG. 2 in detail.

FIG. 5 illustrates an electrical plant monitoring device according to an embodiment of the present disclosure.

FIG. 6 illustrates an operation method of an electrical plant monitoring device according to an embodiment of the present disclosure.

### DETAILED DESCRIPTION

Below, embodiments of the present disclosure will be described in detail and clearly to such an extent that an ordinary one in the art easily carries out the present disclosure.

In the detailed description, components which are described with reference to the terms “unit”, “module”, “block”, “˜er or ˜or”, etc. and function blocks which are illustrated in drawings will be implemented in the form of software or hardware or a combination thereof. For example, the software may include a machine code, firmware, an embedded code, and application software. For example, the hardware may include an electrical circuit, an electronic circuit, a processor, a computer, an integrated circuit, integrated circuit cores, a pressure sensor, an inertial sensor, a microelectromechanical system (MEMS), a passive element, or a combination thereof.

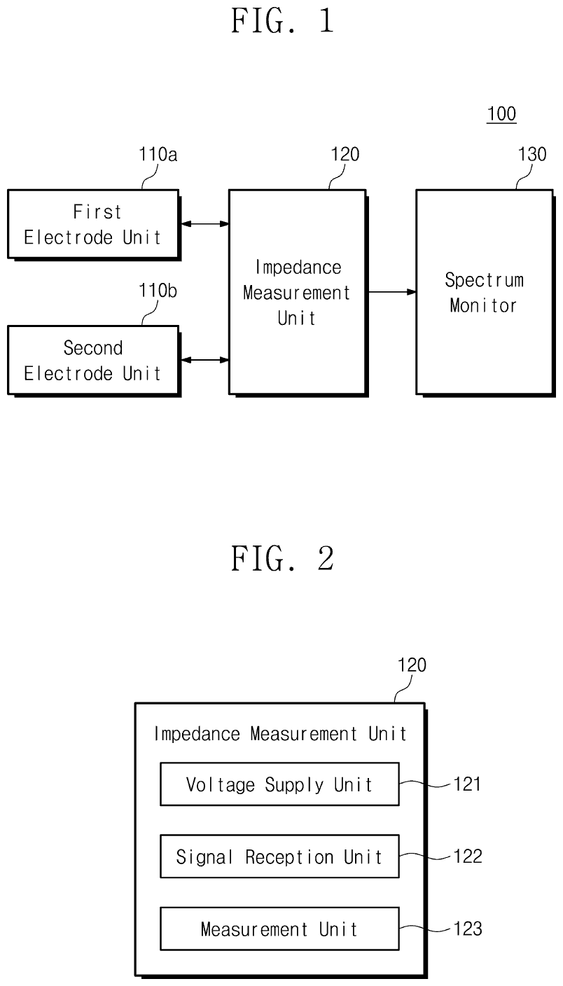

FIG. 1 illustrates an electrical plant monitoring device 100 according to an embodiment of the present disclosure. The electrical plant monitoring device 100 disclosed in FIG. 1 may measure impedance values of plate-shaped and tubular tissues of a plant, may generate an impedance spectrum based on the measured impedance values, and may monitor the impedance spectrum to sense and analyze a change in ions (hereinafter referred to as an “ion stress of a plant”) introduced into the plant.

Referring to FIG. 1, the electrical plant monitoring device 100 may include a first electrode unit 110a, a second electrode unit 110b, an impedance measurement unit 120, and a spectrum monitor 130.

The first electrode unit 110a may include a plurality of flexible electrodes. The plurality of flexible electrodes included in the first electrode unit 110a may be attached to the plate-shaped tissue of the plant. The plurality of flexible electrodes included in the first electrode unit 110a may be formed of a flexible material so as to be attached to a curved surface of the plate-shaped tissue of the plant. For example, the plurality of flexible electrodes included in the first electrode unit 110a may be attached to the leaf of the plant.

The plurality of flexible electrodes included in the first electrode unit 110a may be directly connected to the impedance measurement unit 120 through electrical cables. The plurality of flexible electrodes included in the first electrode unit 110a may exchange various electrical signals with the impedance measurement unit 120 through the electrical cables.

For example, the plurality of flexible electrodes included in the first electrode unit 110a may be supplied with a voltage from the impedance measurement unit 120 through the electrical cables. Based on the supplied voltage, the plurality of flexible electrodes included in the first electrode unit 110a may receive electrical signals from the plate-shaped tissue of the plant and may transfer the obtained electrical signals to the impedance measurement unit 120.

The second electrode unit 110b may include a plurality of flexible electrodes. The plurality of flexible electrodes included in the second electrode unit 110b may be attached to the tubular tissue of the plant. The plurality of flexible electrodes included in the second electrode unit 110b may be formed of a flexible material so as to be attached the tubular tissue of the plant in a wound shape. For example, the plurality of flexible electrodes included in the second electrode unit 110b may be attached to the stem of the plant.

The plurality of flexible electrodes included in the second electrode unit 110b may be directly connected to the impedance measurement unit 120 through electrical cables. The plurality of flexible electrodes included in the second electrode unit 110b may exchange various electrical signals with the impedance measurement unit 120 through the electrical cables.

For example, the plurality of flexible electrodes included in the second electrode unit 110b may be supplied with a voltage from the impedance measurement unit 120 through the electrical cables. Based on the supplied voltage, the plurality of flexible electrodes included in the second electrode unit 110b may receive electrical signals from the tubular tissue of the plant and may transfer the obtained electrical signals to the impedance measurement unit 120.

The impedance measurement unit 120 may obtain the electrical signals associated with the plate-shaped tissue and the tubular tissue of the plant by using the first electrode unit 110a and the second electrode unit 110b. For example, the impedance measurement unit 120 may supply the voltage to each of the first electrode unit 110a and the second electrode unit 110b. Based on the supplied voltage, the first electrode unit 110a may receive the electrical signals from the plate-shaped tissue of the plant and may transfer the received electrical signals to the impedance measurement unit 120. Based on the supplied voltage, the second electrode unit 110b may receive the electrical signals from the tubular tissue of the plant and may transfer the received electrical signals to the impedance measurement unit 120. The impedance measurement unit 120 may obtain the electrical signals associated with the plate-shaped tissue and the tubular tissue of the plant by receiving the electrical signals from the first electrode unit 110a and the second electrode unit 110b.

In some embodiments, the impedance measurement unit 120 may adjust the voltage such that the frequency of the voltage to be supplied to the first electrode unit 110a and the second electrode unit 110b changes. In the case of adjusting the voltage to be supplied to the first electrode unit 110a and the second electrode unit 110b, the impedance measurement unit 120 may obtain electrical signals associated with the plate-shaped tissue and the tubular tissue of the plant so as to correspond to the change in the frequency.

The impedance measurement unit 120 may measure impedance values associated with the plate-shaped tissue and the tubular tissue of the plant based on the obtained electrical signals. For example, the impedance measurement unit 120 may measure the impedance values associated with the plate-shaped tissue of the plant, based on the electrical signals received from the first electrode unit 110a. The impedance measurement unit 120 may measure impedance values associated with the tubular tissue of the plant based on the electrical signals received from the second electrode unit 110b.

The impedance measurement unit 120 may transfer the measured impedance values to the spectrum monitor 130.

The spectrum monitor 130 may generate an impedance spectrum based on the impedance values received from the impedance measurement unit 120. The spectrum monitor 130 may monitor the impedance spectrum to sense an ion stress of the plant. For example, the spectrum monitor 130 may sense the ion stress of the plant by analyzing a change in the impedance values (e.g., a magnitude change and a phase angle change in the impedance values) corresponding to a frequency change of the voltage supplied to the first electrode unit 110a and the second electrode unit 110b.

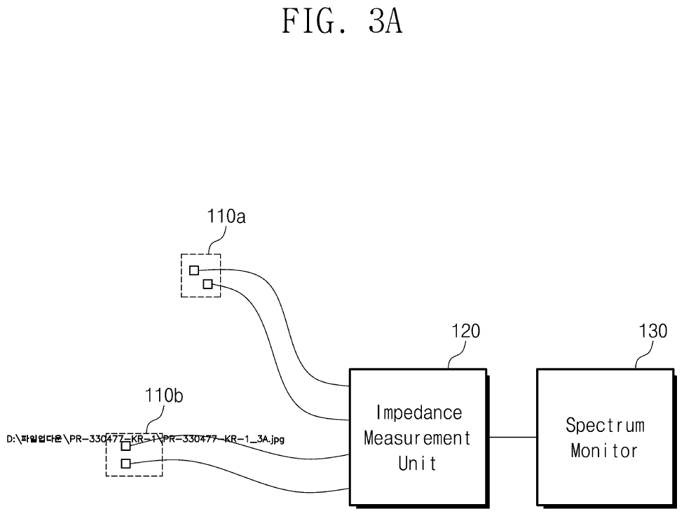

FIG. 2 illustrates the impedance measurement unit 120 of FIG. 1 in detail. Referring to FIGS. 1 and 2, the impedance measurement unit 120 may include a voltage supply unit 121, a signal reception unit 122, and a measurement unit 123.

The voltage supply unit 121 may supply the voltage to each of the first electrode unit 110a and the second electrode unit 110b. For example, the voltage supply unit 121 may supply a first voltage with a first frequency to each of the first electrode unit 110a and the second electrode unit 110b.

The voltage supply unit 121 may adjust a voltage such that the frequency of the voltage to be supplied to each of the first electrode unit 110a and the second electrode unit 110b changes. For example, the voltage supply unit 121 may supply the first voltage with the first frequency to each of the first electrode unit 110a and the second electrode unit 110b and may then supply a second voltage with a second frequency to each of the first electrode unit 110a and the second electrode unit 110b.

The signal reception unit 122 may receive electrical signals from the first electrode unit 110a and the second electrode unit 110b. For example, the signal reception unit 122 may receive, from the first electrode unit 110a, electrical signals based on the first voltage and electrical signals based on the second voltage. In this case, the electrical signals received from the first electrode unit 110a may be electrical signals associated with the plate-shaped tissue of the plant.

For example, the signal reception unit 122 may receive, from the second electrode unit 110b, electrical signals based on the first voltage and electrical signals based on the second voltage. In this case, the electrical signals received from the second electrode unit 110b may be electrical signals associated with the tubular tissue of the plant.

The signal reception unit 122 may transfer the electrical signals received from the first electrode unit 110a and the second electrode unit 110b to the measurement unit 123.

The measurement unit 123 may receive the electrical signals from the signal reception unit 122. The measurement unit 123 may measure the impedance values associated with the plate-shaped tissue and the tubular tissue of the plant, based on the electrical signals.

For example, the measurement unit 123 may measure the impedance values associated with the plate-shaped tissue of the plant, based on the electrical signals received from the first electrode unit 110a. The measurement unit 123 may measure the impedance values associated with the tubular tissue of the plant, based on the electrical signals received from the second electrode unit 110b.

The measurement unit 123 may transfer the measured impedance values to the spectrum monitor 130.

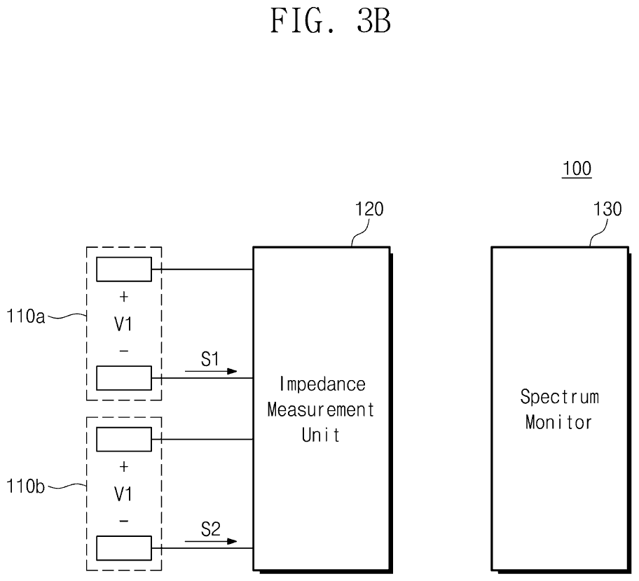

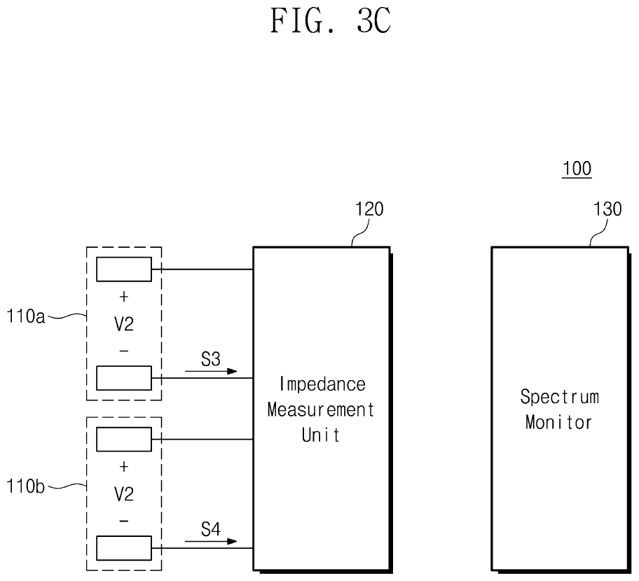

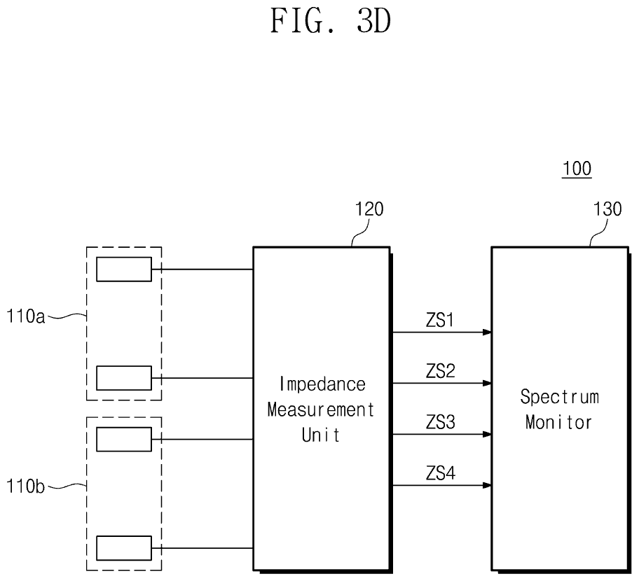

FIGS. 3A to 3D illustrate an example of an operation of the electrical plant monitoring device 100 of FIG. 1. In FIGS. 3 to 3D, it is assumed that the first electrode unit 110a includes a first flexible electrode pair including two flexible electrodes and the second electrode unit 110b includes a second flexible electrode pair including two flexible electrodes. However, the present disclosure is not limited thereto. For example, each of the first electrode unit 110a and the second electrode unit 110b may include a plurality of flexible electrode pairs.

Referring to FIG. 3A, the first flexible electrode pair included in the first electrode unit 110a may be attached to the plate-shaped tissue of the plant such as a leaf, and the second flexible electrode pair may be attached to the tubular tissue of the plant such as a stem.

Referring to FIG. 3B, the impedance measurement unit 120 may supply the first voltage with the first frequency to each of the first electrode unit 110a and the second electrode unit 110b. The impedance measurement unit 120 may receive a first electrical signal S1 associated with the plate-shaped tissue of the plant from the first flexible electrode pair included in the first electrode unit 110a. The impedance measurement unit 120 may receive a second electrical signal S2 associated with the tubular tissue of the plant from the second flexible electrode pair included in the second electrode unit 110b.

Referring to FIG. 3C, after the impedance measurement unit 120 supplies a first voltage V1, the impedance measurement unit 120 may supply a second voltage V2 with the second frequency to each of the first electrode unit 110a and the second electrode unit 110b. The impedance measurement unit 120 may receive a third electrical signal S3 associated with the plate-shaped tissue of the plant from the first flexible electrode pair included in the first electrode unit 110a. The impedance measurement unit 120 may receive a fourth electrical signal S4 associated with the tubular tissue of the plant from the second flexible electrode pair included in the second electrode unit 110b.

Referring to FIGS. 3A to 3D, the impedance measurement unit 120 may measure a first impedance value based on the first electrical signal S1, may measure a second impedance value based on the second electrical signal S2, may measure a third impedance value based on the third electrical signal S3, and may measure a fourth impedance value based on the fourth electrical signal S4. In this case, the first and third impedance values may indicate impedance values between the flexible electrodes of the first flexible electrode pair, and the second and fourth impedance values may indicate impedance values between the flexible electrodes of the second flexible electrode pair.

The impedance measurement unit 120 may transfer first to fourth impedance signals ZS1 to ZS4 respectively including the first to fourth impedance values to the spectrum monitor 130. That is, the impedance measurement unit 120 may transfer the first impedance signal ZS1 including the first impedance value, the second impedance signal ZS2 including the second impedance value, the third impedance signal ZS3 including the third impedance value, and the fourth impedance signal ZS4 including the fourth impedance value to the spectrum monitor 130.

In some embodiments, the impedance measurement unit 120 may transfer the first to fourth impedance signals ZS1 to ZS4 to the spectrum monitor 130 simultaneously or sequentially. For example, the impedance measurement unit 120 may measure the first impedance value and the second impedance value and may then transfer the first impedance signal and the second impedance signal to the spectrum monitor 130. Afterwards, the impedance measurement unit 120 may measure the third impedance value and the fourth impedance value and may then transfer the third impedance signal and the fourth impedance signal to the spectrum monitor 130.

The spectrum monitor 130 may receive the first to fourth impedance signals ZS1 to ZS4. The spectrum monitor 130 may generate an impedance spectrum, based on the first to fourth impedance values included in the first to fourth impedance signals ZS1 to ZS4.

The spectrum monitor 130 may monitor the impedance spectrum to sense an ion stress of the plant.

For example, the spectrum monitor 130 may compare the first impedance value and the third impedance value. As a comparison result, the spectrum monitor 130 may sense the ion stress in the plate-shaped tissue of the plant by analyzing a difference of the first and third impedance values due to a difference of the first frequency and the second frequency (e.g., by analyzing a magnitude difference and a phase angle difference between the first impedance value and the third impedance value).

For example, the spectrum monitor 130 may compare the second impedance value and the fourth impedance value. As a comparison result, the spectrum monitor 130 may sense the ion stress in the tubular tissue of the plant by analyzing a difference of the second and fourth impedance values due to a difference of the first frequency and the second frequency (e.g., by analyzing a magnitude difference and a phase angle difference between the second impedance value and the fourth impedance value).

For example, the spectrum monitor 130 may compare the first impedance value and the second impedance value (or the third impedance value and the fourth impedance value). As a comparison result, the spectrum monitor 130 may infer a transfer path of the ion stress of the plant. That is, the spectrum monitor 130 may infer whether the ion stress of the plant is transferred from the root to the leaf or from the leaf to the root.

In FIGS. 3A to 3D, the description is given as the electrical plant monitoring device 100 senses the ion stress of the plant based on a first voltage V1 and a second voltage V2, but the present disclosure is not limited thereto. For example, the electrical plant monitoring device 100 may sense the ion stress of the plant based on voltages with various frequencies.

As described above, the electrical plant monitoring device 100 may sense the ion stress of the plant in real time based on the voltage supplied to each of the first electrode unit 110a and the second electrode unit 110b. The electrical plant monitoring device 100 may sense and analyze various stresses affecting the plant, such as a drought stress, based on the sensed ion stress.

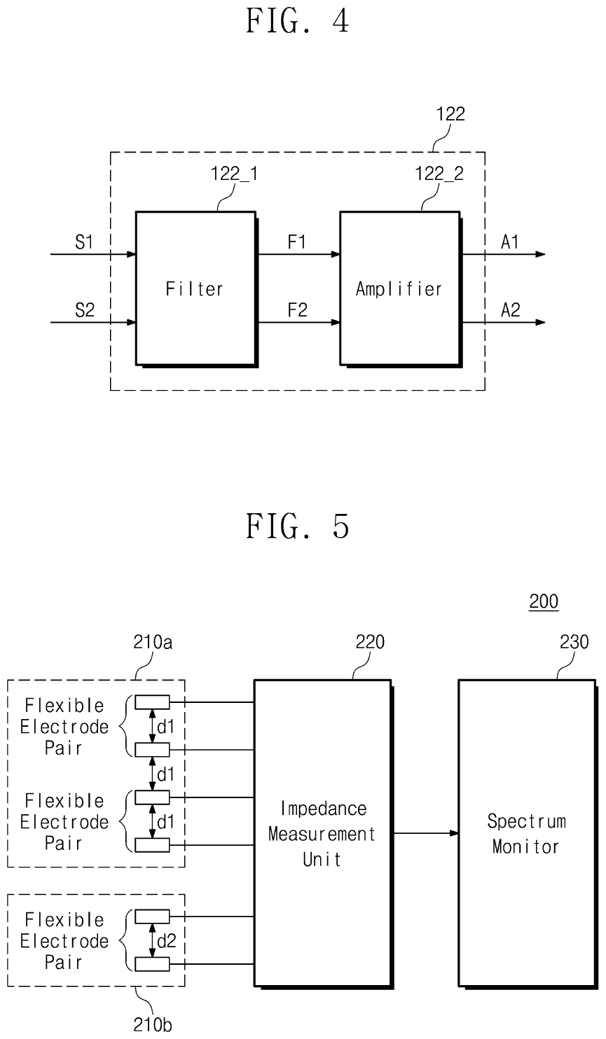

FIG. 4 illustrates the signal reception unit 122 of FIG. 2 in detail. Referring to FIGS. 1, 2, and 4, the signal reception unit 122 may include a filter 122_1 and an amplifier 122_2.

The filter 122_1 may receive the first electrical signal S1 from the first electrode unit 110a and may receive the second electrical signal S2 from the second electrode unit 110b. The filter 122_1 may perform a filtering operation on the received electrical signals S1 and S2.

For example, the filter 122_1 may perform the filtering operation of passing or blocking only a signal in a specific bandwidth in association with the electrical signals S1 and S2. The filter 122_1 may filter the first electrical signal S1 to output a first filter signal F1 and may filter the second electrical signal S2 to output a second filter signal F2.

The amplifier 122_2 may receive the first and second filter signals F1 and F2 from the filter 122_1. The amplifier 122_2 may respectively amplify the first and second filter signals F1 and F2 to output first and second amplification signals A1 and A2.

In some embodiments, the amplifier 122_2 may be implemented in the form of a differential amplifier amplifying a difference of a positive input and a negative input.

FIG. 5 illustrates an electrical plant monitoring device 200 according to an embodiment of the present disclosure. In FIG. 5, operations of an impedance measurement unit 220 and a spectrum monitor 230 are the same as the operations of the impedance measurement unit 120 and the spectrum monitor 130 of FIG. 1, and thus, additional description will be omitted to avoid redundancy.

Referring to FIGS. 1 and 5, a first electrode unit 210a may include four flexible electrodes constituting two flexible electrode pairs. The four flexible electrodes included in the first electrode unit 210a may be attached to the plate-shaped tissue of the plant so to be spaced apart from each other as much as a first distance d1. That is, the first electrode unit 210a may be implemented in an array structure in which flexible electrodes are arranged to be spaced apart from each other as much as the first distance d1.

When the first electrode unit 210a is attached to the leaf of the plant, the spectrum monitor 230 may monitor the impedance spectrum to infer the transfer path of the ion stress. For example, the spectrum monitor 230 may infer the transfer path of the ion stress by distinguishing the ion stress spread along the vein of the plant from the ion stress spread regardless of the vein.

When the first distance d1 of the flexible electrodes included in the first electrode unit 210a is adjusted, the spectrum monitor 230 may monitor the impedance spectrum to analyze a change in an impedance value according to the change in the first distance d1. As an analysis result, the spectrum monitor 230 may infer the transfer path of the ion stress in the plate-shaped tissue of the plant.

A second electrode unit 210b may include two flexible electrodes constituting a flexible electrode pair. The two flexible electrodes included in the second electrode unit 210b may be attached to the tubular tissue of the plant so to be spaced apart from other as much as a second distance d2.

When the second distance d2 of the flexible electrodes included in the second electrode unit 210b is adjusted, the spectrum monitor 230 may monitor the impedance spectrum to analyze a change in an impedance value according to the change in the second distance d2. As an analysis result, the spectrum monitor 230 may infer the transfer path of the ion stress in the tubular tissue of the plant.

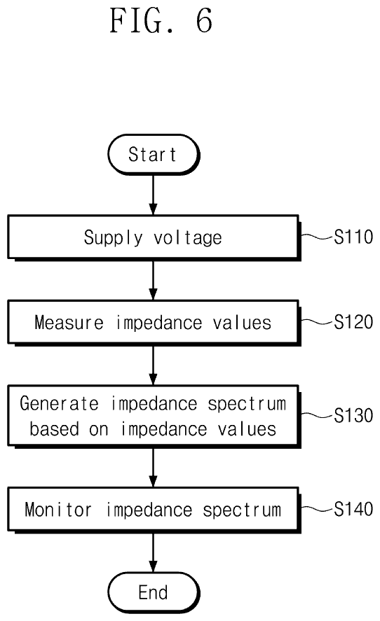

FIG. 6 illustrates an operation method of the electrical plant monitoring device 100 according to an embodiment of the present disclosure. Referring to FIGS. 1, 3A to 3D, and 6, the electrical plant monitoring device 100 may perform operation S110 to operation S140.

In operation S110, the impedance measurement unit 120 may supply the voltage to each of the first electrode unit 110a and the second electrode unit 110b. For example, the impedance measurement unit 120 may supply the first voltage V1 with the first frequency to each of the first electrode unit 110a and the second electrode unit 110b.

In some embodiments, the impedance measurement unit 120 may adjust the voltage which is supplied to each of the first electrode unit 110a and the second electrode unit 110b. For example, the impedance measurement unit 120 may supply the first voltage V1 with the first frequency to each of the first electrode unit 110a and the second electrode unit 110b and may then supply the second voltage V2 with the second frequency to each of the first electrode unit 110a and the second electrode unit 110b.

In operation S120, the impedance measurement unit 120 may measure impedance values, based on the voltages supplied to the first electrode unit 110a and the second electrode unit 110b.

For example, the impedance measurement unit 120 may receive the first electrical signal S1, which is based on the first voltage V1, from the first electrode unit 110a. The impedance measurement unit 120 may measure a first impedance value based on the first electrical signal S1.

For example, the impedance measurement unit 120 may receive the second electrical signal S2, which is based on the first voltage V1, from the second electrode unit 110b. The impedance measurement unit 120 may measure a second impedance value based on the second electrical signal S2.

For example, the impedance measurement unit 120 may receive the third electrical signal S3, which is based on the second voltage V2, from the first electrode unit 110a. The impedance measurement unit 120 may measure a third impedance value based on the third electrical signal S3.

For example, the impedance measurement unit 120 may receive the fourth electrical signal S4, which is based on the second voltage V2, from the second electrode unit 110b. The impedance measurement unit 120 may measure a fourth impedance value based on the fourth electrical signal S4.

In operation S130, the spectrum monitor 130 may generate an impedance spectrum, based on the impedance values measured from the impedance measurement unit 120. For example, the spectrum monitor 130 may receive the first to fourth impedance signals ZS1 to ZS4, in which the first to fourth impedance values are respectively included, from the impedance measurement unit 120 and may generate the impedance spectrum based on the first to fourth impedance values.

In operation S140, the spectrum monitor 130 may monitor the impedance spectrum. As a monitoring result, the spectrum monitor 130 may sense the ion stress of the plant.

For example, the spectrum monitor 130 may monitor the impedance spectrum to analyze a difference between the first and third impedance values due to the difference of the first frequency and the second frequency. As a monitoring result, the spectrum monitor 130 may sense the ion stress in the plate-shaped tissue of the plant.

For example, the spectrum monitor 130 may monitor the impedance spectrum to analyze a difference between the second and fourth impedance values due to the difference of the first frequency and the second frequency. As a monitoring result, the spectrum monitor 130 may sense the ion stress in the tubular tissue of the plant.

For example, the spectrum monitor 130 may monitor the impedance spectrum to analyze a difference between the first and second impedance values (or a difference of the third and fourth impedance values) due to a difference between plant tissues. As a monitoring result, the spectrum monitor 130 may infer the transfer path of the ion stress of the plant.

In the above embodiments, components according to the present disclosure are described by using the terms “first”, “second”, “third”, etc. However, the terms “first”, “second”, “third”, etc. may be used to distinguish components from each other and do not limit the present disclosure. For example, the terms “first”, “second”, “third”, etc. do not involve an order or a numerical meaning of any form.

According to the present disclosure, as flexible electrodes are directly connected to the leaf or stem of the plant, the damage and variable due to the flexible electrodes may be minimized, thus making long-term measurement possible.

According to the present disclosure, various stresses such as an ion stress and a drought stress may be independently monitored by analyzing impedance and phase angle changes in a wide frequency range from various angles.

While the present disclosure has been described with reference to embodiments thereof, it will be apparent to those of ordinary skill in the art that various changes and modifications may be made thereto without departing from the spirit and scope of the present disclosure as set forth in the following claims.

What is claimed is:

1. An electrical plant monitoring device comprising:
a plurality of flexible electrodes attached to a plate-shaped tissue and a tubular tissue of a plant;
an impedance measurement unit configured to obtain electrical signals by using the plurality of flexible electrodes and to measure impedance values associated with the plate-shaped tissue and the tubular tissue based on the electrical signals; and
a spectrum monitor configured to generate an impedance spectrum based on the impedance values and to monitor the impedance spectrum to sense an ion stress of the plant.

2. The electrical plant monitoring device of claim 1, wherein the plurality of flexible electrodes include:
a first flexible electrode pair attached to the plate-shaped tissue; and
a second flexible electrode pair attached to the tubular tissue, and
wherein the impedance measurement unit is configured to:
obtain a first electrical signal among the electrical signals by using the first flexible electrode pair; and
obtain a second electrical signal among the electrical signals by using the second flexible electrode pair.

3. The electrical plant monitoring device of claim 2, wherein the impedance measurement unit is configured to:
measure a first impedance value between flexible electrodes of the first flexible electrode pair from among the impedance values, based on the first electrical signal; and
measure a second impedance value between flexible electrodes of the second flexible electrode pair from among the impedance values, based on the second electrical signal.

4. The electrical plant monitoring device of claim 3, wherein the impedance measurement unit includes:
a voltage supply unit configured to supply a voltage to each of the first flexible electrode pair and the second flexible electrode pair;
a signal reception unit configured to receive the first electrical signal, which is based on the voltage, from the first flexible electrode pair and to receive the second electrical signal, which is based on the voltage, from the second flexible electrode pair; and
a measurement unit configured to measure the first impedance value based on the first electrical signal and to measure the second impedance value based on the second electrical signal.

5. The electrical plant monitoring device of claim 4, wherein the signal reception unit includes:
a filter configured to remove a noise included in the first electrical signal and the second electrical signal and to output a first filter signal and a second filter signal which are noise-free; and
an amplifier configured to amplify the first filter signal and the second filter signal respectively to output a first amplification signal and a second amplification signal, and
wherein the measurement unit measures the first impedance value from the first amplification signal and measures the second impedance value from the second amplification signal.

6. The electrical plant monitoring device of claim 4, wherein the voltage supply unit adjusts the voltage such that a frequency of the voltage changes, and
wherein, as a result of monitoring the impedance spectrum, the spectrum monitor senses the ion stress by analyzing a change in the first impedance value and a change in the second impedance value corresponding to the change in the frequency.

7. The electrical plant monitoring device of claim 2, wherein the plurality of flexible electrodes further include a third flexible electrode pair attached to the plate-shaped tissue,
wherein the impedance measurement unit obtains a third electrical signal among the electrical signals by using the third flexible electrode pair, and
wherein flexible electrodes included in the first flexible electrode pair and the third flexible electrode pair are arranged to be spaced apart from each other by a given distance.

8. The electrical plant monitoring device of claim 7, wherein a transfer path of the ion stress is inferred based on the first flexible electrode pair and the third flexible electrode pair.

9. The electrical plant monitoring device of claim 2, wherein a transfer path of the ion stress is inferred by adjusting a distance of flexible electrodes of the second flexible electrode pair.

10. An operation method of an electrical plant monitoring device, the method comprising:
supplying, at an impedance measurement unit, a voltage to a plurality of flexible electrodes attached to a plate-shaped tissue and a tubular tissue of a plant;
measuring, at the impedance measurement unit, impedance values associated with the plate-shaped tissue and the tubular tissue based on the voltage;
generating, at a spectrum monitor, an impedance spectrum based on the impedance values; and
monitoring, at the spectrum monitor, the impedance spectrum to sense an ion stress of the plant.

11. The method of claim 10, wherein the plurality of flexible electrodes include:
a first flexible electrode pair attached to the plate-shaped tissue; and
a second flexible electrode pair attached to the tubular tissue.

12. The method of claim 11, wherein the measuring of the impedance values associated with the plate-shaped tissue and the tubular tissue based on the voltage at the impedance measurement unit includes:
measuring, at the impedance measurement unit, a first impedance value between flexible electrodes of the first flexible electrode pair by using the first flexible electrode pair; and
measuring, at the impedance measurement unit, a second impedance value between flexible electrodes of the second flexible electrode pair by using the second flexible electrode pair.

13. The method of claim 12, wherein the measuring of the first impedance value between the flexible electrodes of the first flexible electrode pair by using the first flexible electrode pair at the impedance measurement unit includes:
obtaining, at the impedance measurement unit, a first electrical signal by using the first flexible electrode pair; and
measuring, at the impedance measurement unit, the first impedance value based on the first electrical signal, and
wherein the measuring of the second impedance value between the flexible electrodes of the second flexible electrode pair by using the second flexible electrode pair at the impedance measurement unit includes:
obtaining, at the impedance measurement unit, a second electrical signal by using the second flexible electrode pair; and
measuring, at the impedance measurement unit, the second impedance value based on the second electrical signal.

14. The method of claim 13, wherein the obtaining of the first electrical signal by using the first flexible electrode pair at the impedance measurement unit includes:
removing, at the impedance measurement unit, a noise included in the obtained first electrical signal; and
amplifying the noise-removed first electrical signal, and
wherein the obtaining of the second electrical signal by using the second flexible electrode pair at the impedance measurement unit includes:
removing, at the impedance measurement unit, a noise included in the obtained second electrical signal; and
amplifying the noise-removed second electrical signal.

15. The method of claim 10, wherein the supplying of the voltage to the plurality of flexible electrodes attached to the plate-shaped tissue and the tubular tissue of the plant at an impedance measurement unit includes:
supplying, at the impedance measurement unit, a first voltage with a first frequency; and
after supplying the first voltage, supplying, at the impedance measurement unit, a second voltage with a second frequency.

16. The method of claim 15, wherein the measuring of the impedance values associated with the plate-shaped tissue and the tubular tissue based on the voltage at the impedance measurement unit includes:
measuring, at the impedance measurement unit, a first impedance value and a second impedance value, which are based on the first voltage; and
measuring, at the impedance measurement unit, a third impedance value and a fourth impedance value, which are based on the second voltage,
wherein the first impedance value and the third impedance value are associated with the plate-shaped tissue, and
wherein the second impedance value and the fourth impedance value are associated with the tubular tissue.

17. The method of claim 16, wherein the monitoring of the impedance spectrum to sense the ion stress of the plant at the spectrum monitor includes:
analyzing a difference of the first impedance value and the third impedance value according to a difference of the first frequency and the second frequency; and
analyzing a difference of the second impedance value and the fourth impedance value according to the difference of the first frequency and the second frequency.

18. The method of claim 10, wherein the monitoring of the impedance spectrum to sense the ion stress of the plant at the spectrum monitor includes:
inferring, at the spectrum monitor, a transfer path of the ion stress.

19. The method of claim 18, wherein the inferring of the transfer path of the ion stress at the spectrum monitor includes:
adjusting a distance between the plurality of flexible electrodes.
